(19) United States
(12) Patent Application Publication
(10) Pub. No.: US 2021/0186429 A1
(43) Pub. Date: Jun. 24, 2021

(54) ON-DEMAND INTRAOCULAR PHYSIOLOGICAL SENSOR WITH TRABECULAR BYPASS FLOW

(71) Applicant: GLAUKOS CORPORATION, San Clemente, CA (US)

(72) Inventors: Nicholas Gunn, Newport Beach, CA (US); Cesario Pereira Dos Santos, Newport Beach, CA (US); David S. Haffner, Mission Viejo, CA (US); Andrew Johnson, San Clemente, CA (US)

(21) Appl. No.: 17/128,989

(22) Filed: Dec. 21, 2020

Related U.S. Application Data
(60) Provisional application No. 62/951,687, filed on Dec. 20, 2019.

(51) Int. Cl. A61B 5/00; A61B 3/16; A61B 5/145; A61F 9/007

(52) U.S. Cl. CPC A61B 5/6867; A61B 3/16; A61B 5/14503; A61B 5/14532; A61B 2562/18; A61B 5/6882; A61B 5/0031; A61B 2562/0247; A61F 9/00781

(57) ABSTRACT

Intraocular physiological sensor implants include a physiological sensor and a fluid pathway including a fluid inlet and a fluid outlet. The physiological sensor includes a sensing layer, a coil layer, and an integrated circuit layer. The intraocular pressure sensor provides trabecular bypass flow. The intraocular pressure sensor detects intraocular pressure, to identify patient conditions such as glaucoma.

### PRIORITY CLAIM

This application claims priority to U.S. Provisional Patent Application No. 62/951,687 filed on Dec. 20, 2019, entitled “ON-DEMAND INTRAOCULAR PHYSIOLOGICAL SENSOR WITH TRABECULAR BYPASS FLOW,” the entire contents of which are incorporated by reference herein and relied upon.

### BACKGROUND

### Field

The field of the invention generally relates to implantable physiological sensors with flow capabilities for trabecular meshwork bypass. In particular, embodiments of the invention generally relate to implantable intraocular sensors for measuring physiological characteristics such as intraocular pressure and/or glucose concentration, while simultaneously providing for trabecular bypass flow to reduce intraocular pressure in the anterior chamber of the eye.

### Description of the Related Art

Some diseases, including glaucoma and diabetes, can be more effectively treated if they are diagnosed early and/or monitored effectively. Glaucoma, for example, is a leading cause of blindness. This disease damages the optic nerve in the eye due to elevated intraocular pressure, which can lead to complete vision loss if untreated. The risk of blindness can be reduced, however, if the elevated intraocular pressure is detected early and is appropriately managed. Similarly, diabetes is a serious condition which can be more effectively treated with early-stage detection of elevated blood glucose concentration and appropriate management. Appropriate management of either of these conditions can be improved using enhanced monitoring.

Accordingly, diagnostic physiological sensors have been developed for implantation within the human body in order to monitor physiological characteristics such as intraocular pressure and glucose concentration. Such implantable sensors may be used to effectively diagnose and treat certain physiological conditions.

### SUMMARY

In light of the disclosure herein, and without limiting the scope of the invention in any way, in a first aspect of the present disclosure, which may be combined with any other aspect listed herein unless specified otherwise, an intraocular physiological sensor implant includes a physiological sensor and a fluid pathway including a fluid inlet and a fluid outlet. The physiological sensor includes a sensing layer, a coil layer, and an integrated circuit layer.

In a second aspect of the present disclosure, which may be combined with any other aspect listed herein unless specified otherwise, the physiological sensor comprises a pressure sensor at the sensing layer.

In a third aspect of the present disclosure, which may be combined with any other aspect listed herein unless specified otherwise, the pressure sensor comprises a capacitive pressure sensor.

In a fourth aspect of the present disclosure, which may be combined with any other aspect listed herein unless specified otherwise, the capacitive pressure sensor comprises a flexible diaphragm electrode spaced apart from a counter electrode.

In a fifth aspect of the present disclosure, which may be combined with any other aspect listed herein unless specified otherwise, the coil layer includes a looped coil, configured to communicate with an external reader.

In a sixth aspect of the present disclosure, which may be combined with any other aspect listed herein unless specified otherwise, the physiological sensor communicates physiological readings to the external reader, responsive to the external reader being held proximate to an eye of a patient.

In a seventh aspect of the present disclosure, which may be combined with any other aspect listed herein unless specified otherwise, the fluid inlet is configured to reside in an anterior chamber of an eye, and wherein the fluid outlet is configured to reside in Schlemm's canal of the eye, such that aqueous humor flows from the anterior chamber to Schlemm's canal via the fluid pathway.

In an eighth aspect of the present disclosure, which may be combined with any other aspect listed herein unless specified otherwise, the sensing layer is hermetically sealed to the coil layer, and wherein the coil layer is hermetically sealed to the integrated circuit layer.

In a ninth aspect of the present disclosure, which may be combined with any other aspect listed herein unless specified otherwise, the intraocular physiological sensor implant further includes an anchor that is either coupled to a housing or integrally formed with the housing.

In a tenth aspect of the present disclosure, which may be combined with any other aspect listed herein unless specified otherwise, the anchor is configured to pierce a trabecular meshwork of an eye, such that the intraocular physiological sensor is retained in an anterior chamber of the eye, adjacent to the trabecular meshwork.

In an eleventh aspect of the present disclosure, which may be combined with any other aspect listed herein unless specified otherwise, the anchor further includes a plurality of physical standoffs.

In a twelfth aspect of the present disclosure, which may be combined with any other aspect listed herein unless specified otherwise, the physiological sensor comprises a glucose sensor.

In a thirteenth aspect of the present disclosure, which may be combined with any other aspect listed herein unless specified otherwise, an intraocular physiological sensor implant includes a physiological sensor, configured to sense a pressure and a housing. The physiological sensor includes a sensing layer, a coil layer, and an integrated circuit layer. The physiological sensor is configured to communicate pressure readings to an external reader, responsive to the external reader being held proximate to an eye of a patient.

In a fourteenth aspect of the present disclosure, which may be combined with any other aspect listed herein unless specified otherwise, the physiological sensor comprises a capacitive pressure sensor.

In a fifteenth aspect of the present disclosure, which may be combined with any other aspect listed herein unless specified otherwise, the capacitive pressure sensor comprises a flexible diaphragm electrode spaced apart from a counter electrode.

In a sixteenth aspect of the present disclosure, which may be combined with any other aspect listed herein unless specified otherwise, the coil layer includes a looped coil, configured to communicate with the external reader.

In a seventeenth aspect of the present disclosure, which may be combined with any other aspect listed herein unless specified otherwise, the sensing layer is hermetically sealed to the coil layer, and wherein the coil layer is hermetically sealed to the integrated circuit layer.

In an eighteenth aspect of the present disclosure, which may be combined with any other aspect listed herein unless specified otherwise, the intraocular physiological sensor implant further includes an anchor, wherein the anchor is either coupled to the housing or integrally formed with the housing.

In a nineteenth aspect of the present disclosure, which may be combined with any other aspect listed herein unless specified otherwise, the anchor is configured to pierce a trabecular meshwork of an eye, such that the intraocular physiological sensor is retained in an anterior chamber of the eye, adjacent to the trabecular meshwork.

In a twentieth aspect of the present disclosure, which may be combined with any other aspect listed herein unless specified otherwise, the anchor further includes a plurality of physical standoffs.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not necessarily have to have all of the advantages listed herein. Moreover, it should be noted that the language used in the specification has been selected for readability and instructional purposes, and not to limit the scope of the inventive subject matter.

### BRIEF DESCRIPTION OF THE DRAWINGS

Various embodiments and features of devices, systems, and methods will be described with reference to the following drawings. The drawings, associated descriptions, and specific implementations are provided to illustrate embodiments of the invention and not to limit the scope of the disclosure.

FIGS. 1A-B illustrate front and back perspective views of an intraocular physiological sensor implant.

FIGS. 1C-D illustrate perspective and cross-sectional views of an anchor to be implemented with an intraocular physiological sensor implant.

FIGS. 2A-B illustrate an intraocular physiological sensor implant implanted into the trabecular meshwork of an eye.

FIG. 3 illustrates a measurement network communicating with an intraocular physiological sensor implant.

FIG. 4 illustrates a layer-by-layer perspective view of a microelectromechanical system (MEMS) to be implemented with an intraocular physiological sensor implant.

FIG. 5 illustrates a layer-by-layer cross-sectional view of a pressure sensor to be implemented with an intraocular physiological sensor implant.

FIG. 6 illustrates a layer-by-layer perspective cross-sectional view of a coil layer configuration.

FIG. 7 illustrates a planar view of a coil layer, prior to attachment of an integrated circuit.

FIG. 8 illustrates a planar view of an integrated circuit.

FIGS. 9A-B illustrate layer-by-layer perspective and cross-sectional views of a MEMS system.

FIGS. 10A-B illustrate layer-by-layer cross-sectional views of alternate pressure sensors.

### DETAILED DESCRIPTION

There is a need to effectively monitor intraocular pressure within a patient's eye in order to detect or monitor the progression of glaucoma. Intraocular pressure can be measured non-invasively using, for example, a tonometer. While tonometers have the advantage of being non-invasive, they have the disadvantages of generally being expensive, non-portable, specialized equipment that requires skilled operation. Accordingly, as a practical matter, it is difficult to use a tonometer to effectively monitor intraocular pressure in a patient's eye with a time resolution greater than one measurement every few days or weeks during doctor visits. However, since intraocular pressure can vary significantly over relatively short periods of time, such relatively sparse intraocular pressure measurements may not provide a complete or accurate picture of the patient's risk for, or progression of, glaucoma. It would therefore be advantageous to be able to measure intraocular pressure more often or even continuously. Additionally, measurement of direct hydrostatic pressure inside the eye, such as within the anterior chamber of the eye, provides more accurate pressure readings than typical tonometry measurements, which are affected by the mechanical properties of the eye. Furthermore, it would be advantageous to measure intraocular pressure while simultaneously treating the underlying causes associated with glaucoma, for example. Namely, an implantable intraocular pressure sensor that also provides for aqueous humor drainage to Schlemm's canal may simultaneously monitor intraocular pressure while providing safe reduction of intraocular pressure via trabecular meshwork bypass.

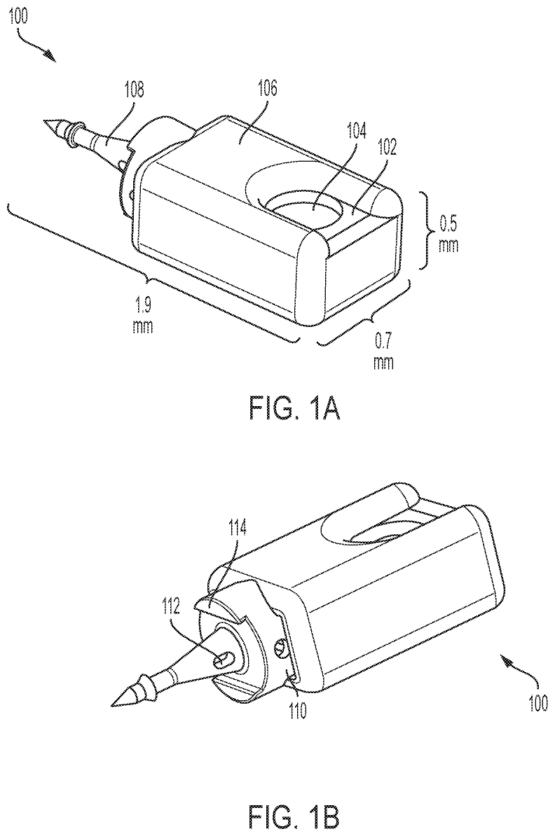

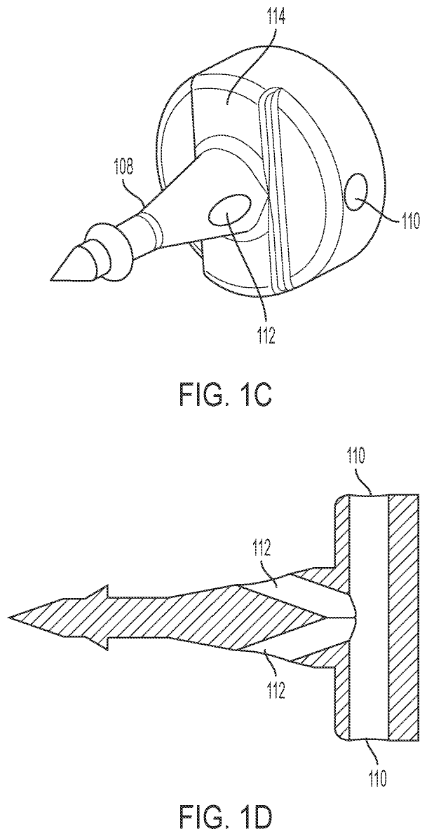

FIGS. 1A and 1B illustrate front and back perspective views of an intraocular physiological sensor implant 100. The intraocular physiological sensor implant 100, referred to herein as sensor implant 100, further includes a pressure sensor 102 with a sensing port 104. While the mechanics of pressure sensor 102 are described in greater detail herein, it should be appreciated that pressure sensor 102 generally communicates with its external environment, such as the anterior chamber of the eye, via sensing port 104. For example, aqueous humor in the anterior chamber of the eye is in contact with sensing port 104; pressure sensor 102 measures the intraocular pressure within the anterior chamber of the eye via fluidic contact with sensing port 104.

Sensor implant 100 further includes protective housing 106. Protective housing 106 surrounds at least a portion of pressure sensor 102, while simultaneously exposing at least a portion of sensing port 104 to the external environment. In some embodiments, housing 106 has contoured surfaces, such as edges that are rounded, filleted, or chamfered, to ensure more smooth contact surfaces with the eye. The selected materials for the housing 106 can be at least partially transmissive and transparent to radio frequency (RF) electromagnetic radiation. Housing 106 may be formed of any biocompatible material such as ceramic or glass. It should be appreciated, however, that other materials may be implemented. In an embodiment, housing 108 is made from a durable and RF transparent material such as ceramic, glass, sapphire, or other material. In the case of ceramic, housing may be micro-molded and/or machined with yttria-stabilized zirconia, zirconia toughened alumina, or other durable ceramics including, but not limited to other zirconia and alumina ceramics. Housing 106 can be further polished smooth to prevent inadvertent damage to surrounding tissue.

In an embodiment, a thin-film coating may be applied to the housing 108, pressure sensor 102, and/or sensor implant 100. The thin film can be, for example, titanium dioxide. The thin-film coating may be applied using atomic layer deposition (ALD) techniques. For example, the pressure senor 102 can be alternately exposed to different gaseous precursor species. Each of the separate precursor species can react with the surface of the housing pressure sensor 102 in a self-limiting manner such that the reaction terminates once all the reactive sites on the surface have been filled. The reaction of each precursor species can deposit a monolayer of atoms on the surface of the pressure sensor 102. By sequentially exposing the pressure sensor 102 to different precursor species, a perfect crystalline structure can be built up layer by layer. The thin-film ALD coating may be a conformal layer of pinhole-free, crystalline titanium dioxide (TiO₂). Other ALD coatings may be applied in a similar manner, including, but not limited to aluminum oxide (Al₂O₃), silicon dioxide (SiO₂), silicon nitride (Si₃N₄), halfnium dioxide (HfO₂). Additionally, by using appropriate precursor species in appropriate orders, multilayer ALD coatings consisting of two or more of the above materials, or additional materials, may be applied to the surface of pressure sensor 102. Such multilayer coatings may consist of a single layer each of two or more materials, or a multitude of layers such that each material is present as several distinct layers within the entire layer stack up. All of these coating options may be applied to the housing 108, pressure sensor 102, and/or sensor implant 100. An ALD coating such as this can provide several benefits. It can act as an additional hermetic seal to help prevent aqueous humor from penetrating or interacting with the housing 106 and pressure sensor 102. The ALD coating can help prevent aqueous humor from interacting with metals from the metallic seal and dissolving them or reacting with them to produce corrosion, as well as prevent metals in the hermetic seal from leaching into solution. In addition, the ALD coating can protect the housing 106 and/or pressure sensor 102 from dissolution in the aqueous humor over time if the materials used to make the housing and/or pressure sensor 102 are in any way water-soluble. Namely, an ALD coating over the pressure sensor 102 and housing 106 could dramatically reduce the rate of dissolution or even prevent dissolution altogether, thereby helping to avoid the need for periodic re-calibration of the pressure sensor 102. The ALD coating itself can be applied in a low-stress manner, so its presence does not significantly affect the mechanical performance of the pressure sensor 102.

Sensor implant 100 further includes anchor 108. Anchor 108 includes at least a flow inlet 110, a flow outlet 112, and a standoff 114. Generally, anchor 108 is affixed to housing 106 and includes a penetrating tip, which is designed to penetrate ocular tissue, such as the sclera, the trabecular meshwork, and the like, and a barb or other retention feature so as to remain anchored in the tissue after having been inserted. In some embodiments, anchor 108 can be drug eluting anchors, similar to the one illustrated in FIG. 18 of U.S. Patent Publication 2015/0342875 (see accompanying appendix), filed May 28, 2015, and entitled “IMPLANTS WITH CONTROLLED DRUG DELIVERY FEATURES AND METHODS OF USING SAME,” the entire contents of which are hereby incorporated by reference herein. Anchor 108 is typically constructed from a hard and durable material, such as titanium. Anchor 108 is attached to housing 106 by brazing, solder attachment, laser welding, physical attachment, such as press-fit, or other related attachment methods. In an embodiment, the housing 106 and anchor 108 are a single monolithic component constructed of ceramic or some other non-metal, such that the anchor 108 is integrally formed with the housing 106. In an embodiment, the housing 106 and/or anchor 108 are constructed of metal in a frame structure or other configuration that limits the amount of material used with housing 106, to ensure that housing 106 is at least partially transmissive and transparent to radio frequency (RF) electromagnetic radiation.

Generally, housing 106 is configured to at least partially surround pressure sensor 102, such that anchor 108 may be coupled to housing 106. It should be appreciated, however, that in alternate embodiments housing 106 is not needed. Namely, for example, in an alternate embodiment anchor 108 is coupled directly to pressure sensor 102 (including its layers), such as via a clip, brace, or other related features. In another alternate embodiment, anchor 108 is glued or welded directly to one or more layers of pressure sensor 102. In these embodiments, where the housing 106 is not included, each of the layers of pressure sensor 102 are hermetically attached to one another.

Anchor 108 may generally be characterized as a drainage stent. Namely, anchor 108 includes flow inlet 110 and flow outlet 112. Thus, anchor 108 enhances outflow of aqueous humor from the eye, similar to the anchor illustrated in FIG. 18 of U.S. Pat. No. 9,554,940 (see accompanying appendix), filed Mar. 14, 2013, and entitled “SYSTEM AND METHOD FOR DELIVERING MULTIPLE OCULAR IMPLANTS,” the entire contents of which are hereby incorporated by reference herein.

Specifically, anchor 108 may include flow inlet 110, flow outlet 112, and a fluid passageway connecting flow inlet 110 to flow outlet 112. While one flow inlet 110 and one flow outlet 112 are illustrated by FIG. 1B, it should be appreciated that additional inlets and outlets are contemplated in various embodiments. For example, opposing inlet and outlets may be disposed on the back side of sensor implant 100 (and thus not shown by FIG. 1B). In an embodiment, sensor implant includes at least two flow inlets 110 and at least two flow outlets 112, connected by at least one branched fluid passageway.

In an embodiment, once the anchor 108 is inserted through the trabecular meshwork and affixed, the flow inlet 110 resides in the anterior chamber of the eye and the flow outlet 112 resides in Schlemm's canal, such that the anchor 108 conducts fluid from the anterior chamber to Schlemm's canal via the fluid passageway.

In an embodiment in which the anchor 108 is a drug eluting anchor, it serves at least two functions: (1) securing the housing 106 (and entire sensor implant 100) to the ocular tissue; and (2) providing to the eye a slow-release drug elution into the anterior chamber to assist with any ocular medical condition requiring continuous medication, such as improving aqueous outflow and treating glaucoma. One such stand-alone drug eluting anchor is described in U.S. Patent Publication No. 2015/0342875, entitled “IMPLANTS WITH CONTROLLED DRUG DELIVERY FEATURES AND METHODS OF USING SAME,” which is incorporated by reference herein. Although only discussed as a stand-alone drug eluting implant in U.S. Patent Pub. No. 2015/0342875, it should be appreciated that the anchor portion of the drug eluting implant could serve the additional purpose of securing to ocular tissue the intraocular sensor 100 discussed herein or any other desirable ocular implant intended to remain static within the anterior chamber (or any other anatomical portion) of the eye.

Standoff 114 is a physical protrusion disposed between flow inlet 110 and flow outlet 112. For example, as illustrated by FIG. 1B, standoff 114 may be disposed on either side of anchor 108. When sensor implant 100 is implanted into the trabecular meshwork of the eye (as described in greater detail below), there is a concern that the sensor implant 100 may have the potential to limit the outflow of aqueous humor, such as through the “conventional” outflow pathway comprising the trabecular meshwork and Schlemm's canal. For example, when implanted, the sensor implant 100 could collapse Schlemm's canal. Standoff 114 thus is configured to provide a pressure relief at the surface of engagement between a face of anchor 108 and the trabecular meshwork. By reducing pressure at the surface of engagement, the likelihood of collapsing Schlemm's canal is substantially reduced. More generally, standoff 114 ensures that aqueous humor may exit the anterior chamber via the physiological outflow pathways even when the sensor implant 100 is positioned against the trabecular meshwork.

In a different embodiment, as illustrated by FIG. 1C, standoff 114 is disposed in a central portion of anchor 108, thus providing a pressure relief to either side of standoff 114 (as described in greater detail above). As previously noted, anchor 108 may include at least two flow inlets 110 and at least two flow outlets 112. This embodiment is illustrated by FIG. 1D. Namely, two flow inlets 110 are disposed in the anterior chamber, whereas two flow outlets 112 are disposed within Schlemm's canal. The two flow inlets 110 and two flow outlets 112 are connected by an angled fluid passageway.

In some embodiments, standoff 114 and/or anchor 108 can be made out of or include a porous material, such as fritted glass, porous plastic such as polypropylene, polyethylene, porous bonded polymer fibers such as polyethylene, polyester, or other materials that are preferably hydrophilic and can be formed into an open-cell porous structure. Such porous materials provide a plurality of fluid handling capillary or pseudo-capillary structures that enable fluid transfer through the bulk structure of the material itself. For example, anchor 108 could be made of a frit (sintered) material, such as titanium; by being porous, the entire anchor 108 could act as a drainage flow path from the anterior chamber to Schlemm's canal. In an embodiment only a portion of anchor 108, such as a distal portion that transits Schlemm's canal, is porous, whereas the remainder of anchor 108 includes a fluid pathway (as previously described). In an embodiment, standoff 114 can be formed directly onto anchor 108 or housing 106.

As illustrated by FIG. 1A, the entire sensor implant 100 including anchor 108 is approximately 1.9 mm in length, 0.7 mm in width, and 0.5 mm in height. It should be appreciated, however, that smaller and/or larger dimensions are contemplated.

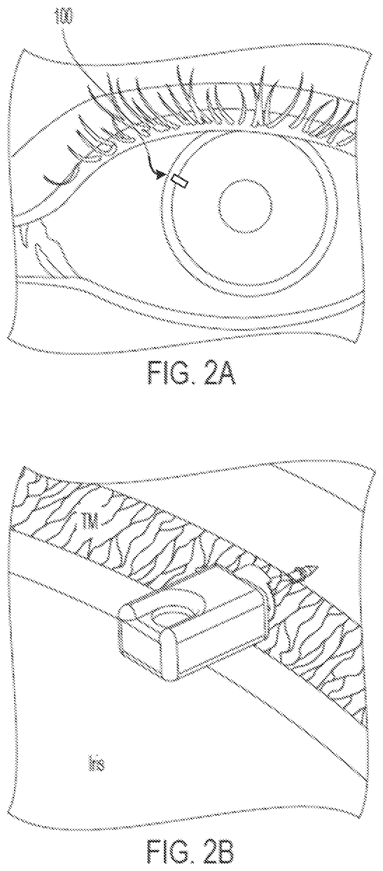

FIGS. 2A and 2B illustrate sensor implant 100, implanted into the trabecular meshwork (TM) of an eye. Sensor implant 100 is implanted in an ab interno injectable surgery. For example, once a surgeon has positioned the sensor implant 100 at the desired location within the patient's eye, he or she exerts a force directed toward the trabecular meshwork or, alternatively, a force is exerted by a specialized inserter device or other surgical tool. This causes the anchor 108 to extend into ocular tissue, so as to hold the housing 106 and entire sensor implant 108 in place. In an embodiment, particular materials of the housing 106 may be configured to “match” the patient's iris. For example, housing 106 may be colored by incorporation of dye, or non dye-including processing techniques such as hot-isostatic pressing (HIPping) or thin film coating such as ALD coating to be more aesthetically camouflaged when implanted. This in turn limits the external visibility of the sensor implant 100. In some embodiments, the sensor implant 100 can be provided with documentation that instructs a surgeon to position the sensor implant 100 at the superior portion of the patient's eye. This position can take advantage of the fact that the upper eyelid typically extends further than the lower eyelid and is therefore able to more effectively hide the sensor implant 100.

While sensor implant 100 is generally described with respect to implantation into the trabecular meshwork (TM) of the eye, in various embodiments, it should be appreciated that the sensors disclosed herein, such as sensor implant 100, could be implanted and positioned in alternate locations within the eye. For example, sensor implant 100 could be implanted into the vitreous cavity, onto or around the retina, onto the intraocular lens, into the iridocorneal angle, and the like. Alternate location implantation can be performed with limited, if any, design changes to the sensor implant 100 disclosed herein. Thus, in certain embodiments, sensor implant 100 is location agnostic.

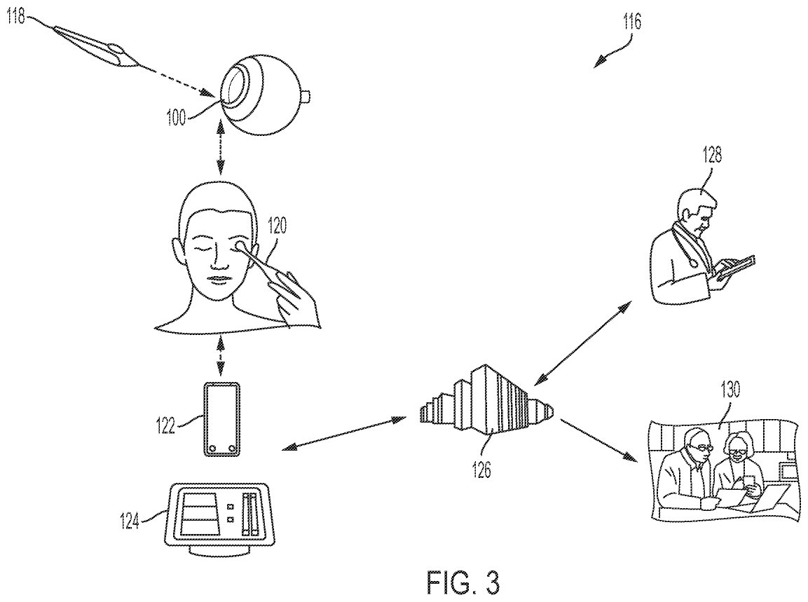

FIG. 3 illustrates a measurement network 116, configured for communicating with sensor implant 100. Namely, as previously noted, sensor implant 100 may be implanted into the eye using a specialized inserter device 118. In an embodiment, the specialized inserter device is a pre-loaded, single-use, device. Once implanted, sensor implant 100 communicates with a reader 120 in an on-demand fashion.

For example, the patient may hold the reader 120, such as a wand or other hand-held device, up to his or her eye; once the reader 120, which includes an inductive coil, is in close enough proximity to the sensor implant 100 (e.g., 5 to 10 mm), the reader 120 inductively powers the sensor implant 100 and wirelessly communicates with the sensor implant 100 to receive a digital readout of the measured capacitance of the pressure sensor 102, which correlates to absolute intraocular pressure. The measured capacitance is converted to a pressure measurement by using a calibration that can be stored on the reader 120 (or accessed remotely, such as via a server). Sensor 100 communicates in an on-demand fashion, in that it only sends a measured capacitance reading when the reader 120 is in close enough proximity; likewise, sensor 100 communicates the instantaneous measured capacitance reading at a particular point in time.

In an embodiment, reader 120 includes audible and/or visual cues to notify the patient as to when the reader 120 is wirelessly communicating with the sensor implant 100. For example, reader 120 can include a speaker that beeps or alarms once the reader 120 has communicated with sensor implant 100. Likewise, for example, reader 120 can include lights, such as red and green lights; in this example, reader 120 displays a red light until it has communicated with sensor 100, at which time it displays a green light. It should be appreciated that other audible and/or visual techniques similar to those discussed above are contemplated herein.

In an embodiment, on-demand communication advantageously reduces any power and/or memory requirements within sensor implant 100. Thus, the overall size of sensor implant 100 is drastically reduced as compared to an implant with autonomous functionality including battery power and/or memory for storing measurement data.

Reader 120 may further include a built-in high-accuracy atmospheric pressure sensor, to measure the ambient environmental pressure at the time of the sensor-reading; this measured pressure can be used in combination with measurements from the sensor implant 100 in order to determine intraocular pressure (IOP), which is defined as the gauge pressure of absolute pressure inside the eye minus local atmospheric pressure. Reader 120 further includes memory, for storing intraocular pressure readings provided by sensor implant 100. In an embodiment, reader 120 has the form factor similar to an eye dropper, providing a degree of familiarity to the patient and facilitating correct positioning of the inductive coil during readings. In an embodiment, reader 120 has the form factor similar to a pair of standard eyeglasses, providing a means for the patient to reliably and repeatably position the reader in relation to their eye and the implant location.

Measurement network 116 may further include a number of additional “downstream” devices and/or systems to handle, display, distribute, and manage patient data, as illustrated in FIG. 3.

For example, reader 120 may transfer received intraocular pressure readings to another external device such as a cellular device 122 or a base station 124. For example, cellular device 122 may run a mobile app that bridges communication between reader 120 and external servers 126. Additionally or alternatively, cellular device 122 may bridge communication between reader 120 and other external devices such as wrist-worn storage devices or smartwatches, which may, likewise, be communicating with reader 120. In various embodiments, the reader 120 communicates with the wrist-worn storage device (or cellular device 122) via Bluetooth, WiFi, Zigbee, or other related wireless communication. It should be appreciated that other external devices, such as bracelets, any other wearable electronic device, cell phones, tablets, e-readers, laptops, and the like are contemplated for communicating with the reader 120.

Cellular device 122 may provide visual information, such as intraocular pressure readings and historical data, to the patient. Base station 124 may, additionally or alternatively, provide similar hardware functionalities as those provided by cellular device 122.

External servers 126 may process and store patient data, such as data wirelessly received from cellular device 122. External servers 126 may further deliver data to portal applications in an on-demand fashion. For example, physician portal 128 provides physicians with an intuitive interface to view patients' intraocular pressure readings, setup alerts, send messages directly to patients and cellular devices 122, and remotely adjust the functionality of sensor implant 100 and/or reader 120. Similarly, for example, patient portal 130 provides patients with simple intraocular pressure measurement tracking information and messages, such as those received from the physician.

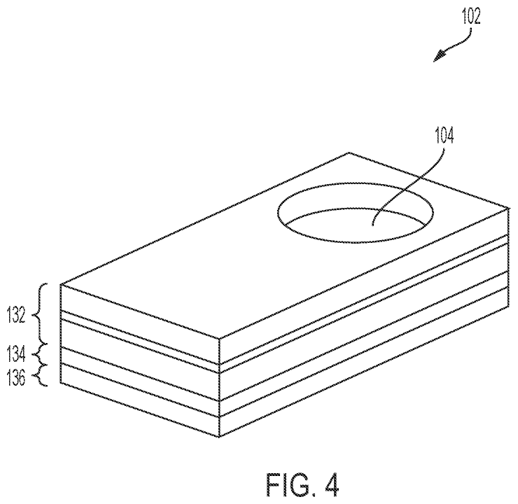

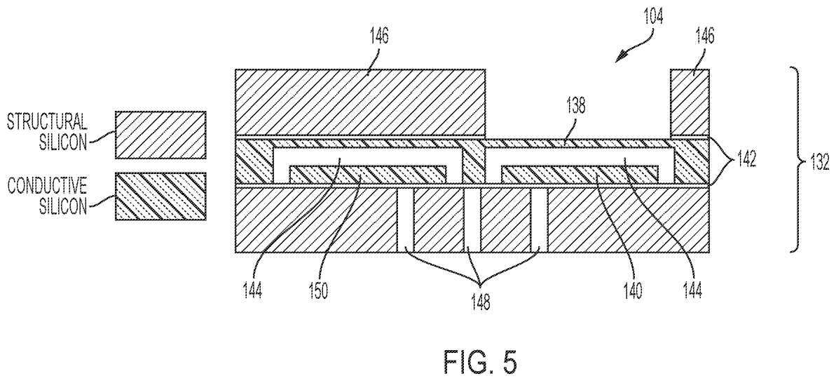

Turning now to the specifics of sensor implant 100, FIGS. 4 and 5 illustrate a layer-by-layer perspective and cross-sectional views of a microelectromechanical system (MEMS), also referred to as pressure sensor 102, to be implemented with sensor implant 100.

Generally speaking, pressure sensor 102 includes three distinct systems: (1) a pressure sensor (along with a reference sensor) that outputs a capacitive signal, (2) an application-specific integrated circuit (ASIC) that converts the capacitive signals to digital representations and communicates with reader 120 by modulating an inductive coil, and (3) an inductive coil that enables the ASIC to receive wireless power from reader 120 and communicate wirelessly with the reader 120. With that in mind, the MEMS device or pressure sensor 102 includes three layers, a pressure sensor layer 132, a coil layer 134, and an integrated circuit layer 136. As illustrated, pressure sensor layer 132 further includes sensing port 104 (as previously discussed).

For example, FIG. 5 illustrates a cross-sectional view of pressure sensor layer 132. Pressure sensor layer 132 generally includes a capacitive pressure sensor fabricated from silicon. The pressure sensor design is essentially a typical parallel plate capacitive pressure sensor in which a flexible membrane electrode 138 is moved closer to a pressure sensor counter electrode 140 when pressure increases, thereby increasing the capacitance of the overall structure.

Both the flexible membrane electrode 138 and pressure sensor counter electrode 140 are made from conductive silicon. In other embodiments the counter electrode is made from a different conductive material, such as gold, platinum, aluminum, or other similar metal. The pressure sensor counter electrode 140 is positioned atop a silicon oxide layer 142 approximately 0.5 to 4 μm thick. The flexible membrane electrode 138 is made from a layer that is thicker in some areas such that it extends down and is positioned atop the same silicon oxide layer 142. A gap 144 of about 0.1 to 1 μm separates the two electrodes and an annular gap surrounds the entire pressure sensor counter electrode 140, such that the two electrodes are isolated. Above the flexible membrane electrode 138 there is another silicon oxide layer 142 approximately 0.5 to 4 μm thick that separates the flexible membrane electrode 138 from a bulk silicon layer 146 above, which provides structure to the pressure sensor 102. In some embodiments, bulk silicon layer 146 is not present. At a portion of the pressure sensor 102 is sensing port 104 in which this bulk silicon layer 146 is removed, and optionally the approximately 0.5 to 4 μm thick silicon oxide layer 142 is also removed, such that the flexible membrane electrode 138 is free-standing and can deflect in response to external pressure. For example, the membrane can deflect responsive to intraocular pressure within the anterior chamber.

Through-silicon vias 148 are provided to connect the flexible membrane electrode 138 and the pressure sensor counter electrode 140. For example, through-silicon vias 148 transit a bottom silicon oxide layer and make electrical contact with each electrode from the underside. In a preferred embodiment, the vias 148 are made from conductive silicon. In other embodiments, the vias 148 may be made from metal. The vias 148 may be solid conductor or, alternatively, predominately open vias with the conductor only on the sidewall of the via. Other structures, such as annular conductor vias, are likewise contemplated in various embodiments. Generally, each of the vias 148 is isolated from the surrounding silicon by an insulator such as silicon oxide, silicon nitride, or another electrically-insulated material such as a polymer. In an embodiment, the bulk silicon layers that are present below and above conductive silicon oxide layers 142 may be instead a different material such as glass or ceramic, or a different material on each side.

In an embodiment, pressure sensor 102 further includes a reference sensor, including a reference sensor counter electrode 150, disposed adjacent to the pressure sensor counter electrode 140. The structure of the reference sensor is similar to that described above with respect to flexible membrane electrode 138 and pressure sensor counter electrode 140, except that the sensing port 104 is not present. Thus, the bulk silicon layer 146 above the reference sensor prevents the flexible membrane electrode 138 in that location, i.e., the portion of flexible membrane electrode 138 that is opposed from reference sensor counter electrode 150, from appreciably responding to pressure changes within the anterior chamber. In this embodiment, a third through-silicon via 148 is used to connect to the reference sensor counter electrode 150. The membrane electrode 138 of the reference sensor is shared with the membrane electrode 138 of the pressure sensor and therefore can share the same via 148 for signal routing when the pressure sensor and reference sensor are measured sequentially. In an alternative embodiment, the membrane electrode 138 is not shared between the pressure sensor and reference sensor, but rather there are two isolated membrane electrodes, one for each sensor. In this embodiment, there is a fourth via required to connect to the second isolated membrane electrode. The capacitive signal generated by the reference sensor counter electrode 150 can be used to filter out effects unrelated to changes in pressure, such as intrinsic stresses, mechanical stresses, temperature changes, and the like, thereby improving the accuracy of the system as a whole. Signal from the reference sensor can be used directly, such as via a direct analogue subtraction of its capacitance from the capacitance of pressure sensor counter electrode 140 and flexible membrane 138 within the ASIC's operation or a digital subtraction of the same, or indirectly by reading its value substantially concurrently with reading the capacitance of pressure sensor counter electrode 140 and flexible membrane 138 and using both measurements during downstream data processing/calibration. In the preferred embodiment, the reference sensor counter electrode 150 has the same dimensions as the pressure sensor counter electrode 140, but can also be of different size and shape and still provide similar utility.

For example, as implemented, pressure sensor layer 132, when combined with the rest of the system of the sensor implant 100, is capable of measuring intraocular pressure from about 0 mmHg to about 50 mmHg of gauge pressure within the eye with about ±0.5 mmHg resolution. In some embodiments, the gap 144 underneath the membrane electrode 138 is sealed under vacuum, and the pressure sensor layer 132 responds over the range of approximately 500 to 1000 mmHg absolute pressure with about an essentially infinite resolution as the output is a variable analogue capacitance signal. The atmospheric pressure can be measured independently outside the body (via a built-in high-accuracy atmospheric pressure sensor disposed within reader 120) and subtracted from the absolute pressure measured by the pressure sensor 102 to yield the intraocular pressure. In some embodiments, the capacitance varies in an approximately linear fashion relative to the intraocular pressure. In some embodiments, the capacitance may increase approximately linearly from approximately 1 picofarads (pF) to approximately 4 pF over the range of absolute pressure from 500 to 1000 mmHg. In other embodiments the absolute pressure range is smaller or larger or the sensitivity of the sensor is more or less. In some embodiments, the capacitance varies in a non-linear fashion relative to intraocular pressure.

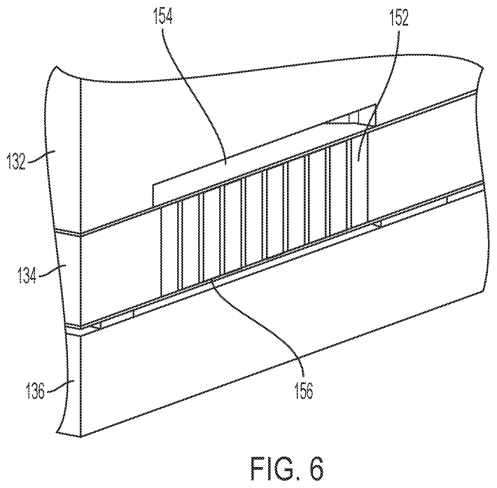

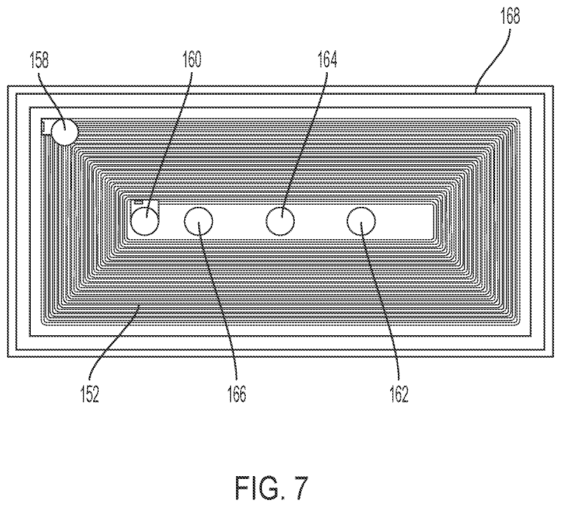

FIGS. 6 and 7 illustrate a layer-by-layer perspective cross-sectional and planar views of a coil layer 134 for pressure sensor 102. Namely, coil layer 134 is contiguous with pressure sensor layer 132 and is adjacent to the via-containing side of pressure sensor layer 132. The coil layer 134 is constructed of silicon and has an integrated inductive coil 152. Alternatively, the coil layer 134 could be of glass, sapphire, or ceramic. In an embodiment, pressure sensor layer 132 is hermetically attached to the coil layer 134 using a silicon fusion bond. In other embodiments, the hermetic attachment is made with a silicon-silicon oxide fusion bond, an anodic bond, metal fusion bond such as Au—Au, or eutectic bond such as Si—Au, In—Au, Sn—Au, or the like.

In an embodiment, integrated inductive coil 152 is a copper coil. In other embodiments, integrated inductive coil 152 is gold, silver, or any other conductive material such as other metals or conductive silicon. Generally, copper is preferred due to its high electrical conductivity, which improves the quality-factor of the coil 152. The cross-sectional dimensions of each turn of the conductor of the coil 152 are approximately 2-10 μm wide by 5-25 μm tall. Between each turn of the coil is an insulator, such as silicon dioxide or glass, that is about 1-4 um wide. The insulator also separates the coil 152 from the bulk silicon. There are approximately 10 to 35 windings of the coil 152 in a planar configuration (as illustrated by FIG. 7). It should be appreciated that the dimensions provided herein relate to one example embodiment of coil 152; other dimensions of the coil, including its cross-section and the total number of windings, are contemplated.

As an alternative to a coil and inductive coupling as described herein, several other alternative wireless transfer technologies are likewise contemplated such as, but not limited to, capacitive coupling, electric-field coupling, and ultrasound. These technologies would substitute the coil (e.g., coil 152) for a conductive plate, an antenna, or a piezoelectric material, respectively. The external reader would also be designed to provide the corresponding powering signal. For capacitive coupling, electric-field, and inductive coupling, the external reader includes an AC signal that is driven into a plate, antenna, or coil, to generate appropriate signal to power the implant. For ultrasonic, the external reader includes a transducer that transmits ultrasonic signals, which would excite the piezoelectric material and consequently generate power for the implant.

There is topology on the adjoining surface of the sensor layer 132, such that when it is hermetically bonded to the coil layer 134, a cavity 154 is created adjacent to the turns of the coil 152. This cavity 154 advantageously reduces parasitic capacitance and the formation eddy currents in the bulk silicon, both of which would otherwise reduce the wireless performance of the inductive coil 152. Likewise, when the integrated circuit layer 136 is joined to the other side of the coil layer 134 (as is described below), a similar cavity 156 is also created adjacent to the turns of the coil 152, due to the height of the metallic hermetic bond between the layers, which also affords the aforementioned benefits.

The side of the coil layer 134 not in contact with the pressure sensor layer 132 includes a number of metal pads (and metal traces that electrically connect the coil 152 to the metal pads), which are appropriately positioned and structured to make electrical connection to the integrated circuit layer 132. For example, coil layer 134 includes a first coil pad 158 and a second coil pad 160. Additionally, the through-silicon vias 148 from the pressure sensor layer 132 transit the coil layer 134 and are also connected to similar metal pads. For example, coil layer 134 includes a pressure sensor electrode pad 162 communicating with pressure sensor counter electrode 140, a membrane sensor electrode pad 164 communicating with membrane electrode 138, and a reference electrode pad 166 communicating with reference sensor counter electrode 150, as illustrated by FIGS. 5 and 7.

In an embodiment, the through-silicon vias 148 are created after the coil layer 134 is joined to the sensor layer 132, such that the through-silicon vias 148 are continuous through both of the pressure sensor layer 132 and the coil layer 134. Alternatively, two distinct sets of vias could be created in each layer, and electrical connections could be made at the interface between the two layers.

The metal pads 162, 164, 166 on the coil layer 134 are positioned such that they make contact to similar metal pads on the integrated circuit layer 136 when these two layers are adjoined face-to-face. Coil layer 134 may further include a seal ring 168, disposed around the entire perimeter of the coil layer 134. Seal ring 168 forms a hermetic seal when the integrated circuit layer 136 is attached to the coil layer 134.

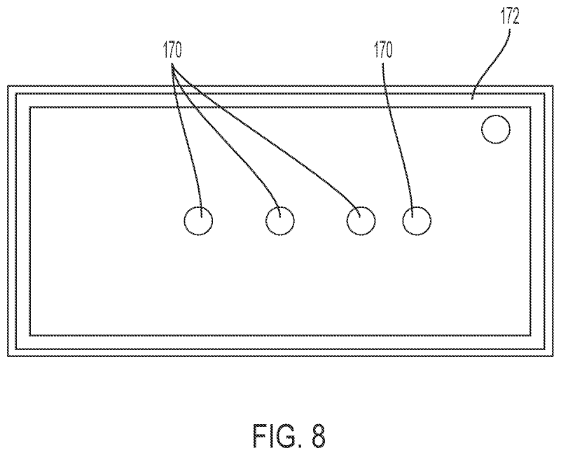

FIG. 8 illustrates a planar view of integrated circuit layer 136, which includes the ASIC. The ASIC itself includes several circuits, including a controller, voltage rectifier, transceiver, capacitance-to-digital converter, non-volatile and/or volatile memory, and the like. The ASIC is fabricated in silicon using complementary metal-oxide-semiconductor (CMOS) fabrication processes. Generally, the ASIC may have an uneven surface topology at the completion of standard CMOS processing. This topology may be on the order of approximately 1 μm, and therefore could interfere with hermetic bonding. Thus, the uneven top-side may be planarized, prior to hermetic bonding, such as by depositing a silicon dioxide layer and then polishing, to create a flat and smooth surface on the top-side. The oxide must be opened at the ASIC contact locations, and a number of metal pads 170 must be formed for connecting to the corresponding pads 160, 162, 164, 166 on the coil layer 134. A metal seal ring 172 is also formed around the perimeter of the integrated circuit layer 136 to match the seal ring 168 on the coil layer 134.

The bond between the coil layer 134 and the integrated circuit layer 136 accomplishes two things. First, it enables the electrical connections between the ASIC and the MEMS components so that the ASIC can measure the sensors and utilize the inductive coil 152 for wireless communication. It also provides for a hermetical seal ring at the perimeter of the part, which isolates the connections from the outside environment, thus preventing crosstalk/leakage and parasitic capacitances. In various embodiments, the bond is an Au—Au fusion bond, a thermo-compression bond, or an eutectic bond such as Si—Au, In—Au, Sn—Au, or other related bonds. In one embodiment a hermetic surface bond is created between coil layer 134 and circuit layer 136 instead of a hermetic seal ring. In this embodiment the bond is continuous across the entire surface, such as a silicon oxide to silicon oxide bond, and the electrical connection transit through the hermetic bond itself as opposed to being surrounded by a hermetic seal ring.

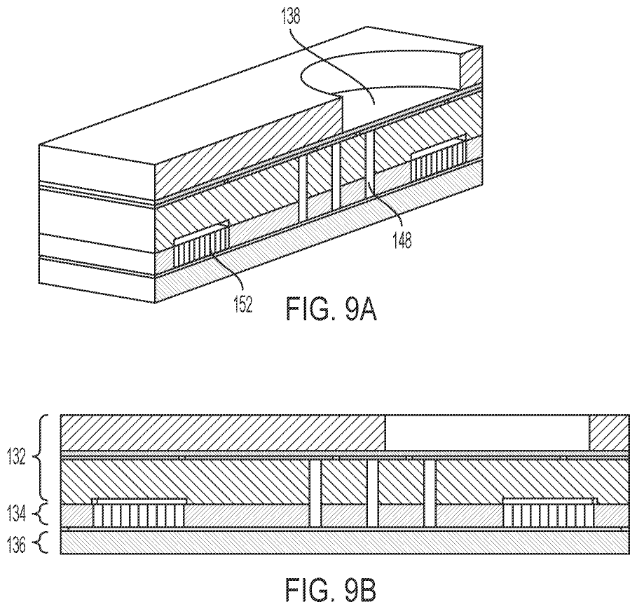

FIGS. 9A and 9B illustrate layer-by-layer perspective cross-sectional and cross-sectional views of a pressure sensor 102 as previously described herein. In summary, the pressure sensor 102 includes the pressure sensor layer 132, which is hermetically bonded to the coil layer 134, which is hermetically bonded to the integrated circuit layer 136. The hermetic bonds can be formed by various bonding methods such as fusion bonding, eutectic bonding, thermo-compression bonding, or related bonding techniques. Because each of the layers 132, 134, 136 are hermetically bonded or sealed with no exposed electrical connections, the entire pressure sensor 102 is hermetically sealed. Thus, housing 106 is not required to be hermetically sealed, as pressure sensor 102 is already protected via its own seals from the external environment.

In a preferred embodiment, the pressure sensor 102 is fabricated at a wafer level, in which the above-described structures are fabricated in batch fabrication in the full wafer format. Only at the completion of the processing are the individual pressure sensors 102 separated (into “dies” or “dice”). The separation may be accomplished using a dicing method, such as blade dicing, laser dicing, or stealth dicing. For example, fabrication at a wafer level helps to ensure part to part consistency and a much lower per-piece cost. However, it is theoretically possible to build the layers 132, 134, 136 separately and perform the hermetic bonding operations at a die-level.

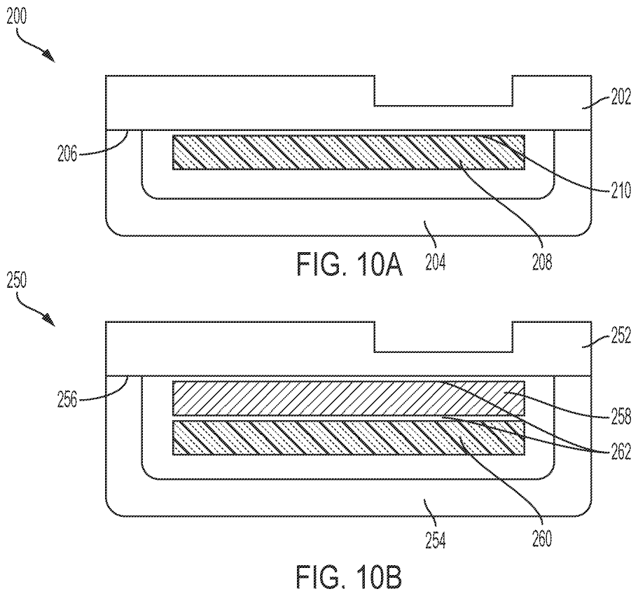

FIGS. 10A and 10B illustrate layer-by-layer cross-sectional views of alternative pressure sensors 200 and 250. For example, FIG. 10A illustrates pressure sensor 200 including combined sensor and coil layers 202, which are attached to housing 204 via hermetic seal 206. Combined sensor and coil layers 202 are similar to pressure sensor layer 132 and coil layer 134 described above. Pressure sensor 200 further includes integrated circuit layer 208, which is attached to sensor and coil layers 202 via a non-hermetic connection 210, such as a conductive adhesive bond. In this embodiment, though sensor layer and coil layer 202 are not hermetically bonded to integrated circuit layer 208, the entire pressure sensor 200 is nonetheless hermetically sealed via housing 204 and hermetic seal 206.

Similarly, for example, FIG. 10B illustrates a pressure sensor 250 including sensor layer 252, which is attached to housing 254 via hermetic seal 256. Pressure sensor 250 further includes coil layer 258 and integrated circuit layer 260, each of which is attached to other layers via a non-hermetic connection 262, such as a conductive adhesive bond. In this embodiment, though none of sensor layer 252, coil layer 258, and integrated circuit layer 260 are hermetically bonded, the entire pressure sensor 250 is nonetheless hermetically sealed via housing 254 and hermetic seal 256.

While the embodiments of the sensor implant 100 described above relate to the measurement of intraocular pressure, it should be appreciated that some embodiments may additionally, or alternatively, measure glucose concentration in the aqueous humor. Specifically, there is a need to measure glucose concentration within the human body as a means to treat or prevent complications from diabetes. Glucose is typically measured from blood or urine sampling. Some implantable glucose sensors have been developed that measure glucose from interstitial fluids; however, the body may have a negative immunological response to such implants, which may degrade the performance of the sensor over time. The eye, especially the anterior chamber of the eye, is an immunologically-privileged site within the body. Thus, an intraocular sensor implant for measuring glucose within the eye can have advantages over other implantable sensors that are made to measure glucose in non-immunologically privileged parts of the body. In addition, although the glucose concentration within the aqueous humor may not be identical to blood glucose concentration, the two may be correlated such that a measurement of glucose concentration in the aqueous humor can be predictive of blood glucose concentration.

In an embodiment, the sensor implant 100 runs a current and measures an initial rate of decay of reactive buildup on a glucose sensor. The reactive buildup may be a buildup of glucose itself or another chemical species related to the measurement of glucose, such as hydrogen peroxide. From this measured initial rate of decay, the sensor implant 100 implements an algorithm to approximate glucose levels at the time at which all buildup would have decayed had the sensor run a current for a longer period of time. In this way, sensor 100 advantageously avoids unnecessary power consumption associated with burning off the entire reactive buildup. Sensor may additionally communicate detected glucose levels in an on-demand fashion with reader 120, as previously described above.

It may be advantageous to measure both intraocular pressure and glucose concentration in the aqueous humor because the glucose concentration measurement can be used to diagnose and/or treat diabetes. Meanwhile, diabetes patients are also at higher risk of developing glaucoma. Thus, there may be a significant overlap of the patient population for whom intraocular pressure and glucose concentration measurements would be valuable.

Furthermore, in various embodiments, it should be appreciated that the sensors disclosed herein, such as sensor implant 100, could be implanted and positioned in alternate locations within the human body. For example, sensor implant 100 could measure pressure of other organs, such as the brain or heart, or other locations, such as the thoracic cavity, intracranial space, exterior limbs, and the like. Likewise, sensor implant 100 could measure other values, such as glucose concentration, at these alternate locations. Alternate location implantation can be performed with limited, if any, design changes to the sensor implant 100 disclosed herein. Thus, in certain embodiments, sensor implant 100 is location agnostic.

Various embodiments of implantable physiological sensors, and associated methods, with a variety of features, have been described herein. Although not every embodiment has been illustrated with every feature, it should be understood that the features described herein can be freely combined with the various embodiments that are described and illustrated. The various physiological sensors described herein can also have any feature, characteristic, element, or the like that is disclosed in connection with the sensor devices described in the following U.S. patent documents, which are each hereby incorporated by reference in their entirety: U.S. Pat. Nos. 6,981,958; 7,678,065; 8,142,364; and U.S. Patent Publication No. 2010/0056979. In addition, the various physiological sensors described herein can be used in, for example, any manner or application that is described in the foregoing patent documents.

The various illustrative devices, logical blocks, modules, circuits, and algorithm steps described in connection with the embodiments disclosed herein can be implemented as, for example, electronic hardware, such as analog and/or digital circuitry, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

Some of the various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein.

Embodiments have been described in connection with the accompanying drawings. However, it should be understood that the figures are not necessarily drawn to scale. Distances, angles, and other dimensions are merely illustrative and do not necessarily bear an exact relationship to actual dimensions and layout of the devices illustrated. In addition, the foregoing embodiments have been described at a level of detail to allow one of ordinary skill in the art to make and use the devices, systems, and methods described herein. A wide variety of variation is possible. Components, elements, and/or steps can be altered, added, removed, or rearranged. While certain embodiments have been explicitly described, other embodiments will become apparent to those of ordinary skill in the art based on this disclosure. The scope of certain inventions disclosed herein is indicated by the appended claims rather than by the foregoing description. All changes which come within the meaning and range of equivalency of the claims are to be embraced within their scope.

What is claimed is:

1. An intraocular physiological sensor implant comprising:
a physiological sensor; and
a fluid pathway including a fluid inlet and a fluid outlet,
wherein the physiological sensor includes a sensing layer, a coil layer, and an integrated circuit layer.

2. The intraocular physiological sensor implant of claim 1, wherein the physiological sensor comprises a pressure sensor at the sensing layer.

3. The intraocular physiological sensor implant of claim 2, wherein the pressure sensor comprises a capacitive pressure sensor.

4. The intraocular physiological sensor implant of claim 3, wherein the capacitive pressure sensor comprises a flexible diaphragm electrode spaced apart from a counter electrode.

5. The intraocular physiological sensor implant of claim 1, wherein the coil layer includes a looped coil, configured to communicate with an external reader.

6. The intraocular physiological sensor implant of claim 5, wherein the physiological sensor communicates physiological readings to the external reader, responsive to the external reader being held proximate to an eye of a patient.

7. The intraocular physiological sensor implant of claim 1, wherein the fluid inlet is configured to reside in an anterior chamber of an eye, and wherein the fluid outlet is configured to reside in Schlemm's canal of the eye, such that aqueous humor flows from the anterior chamber to Schlemm's canal via the fluid pathway.

8. The intraocular physiological sensor implant of claim 1, wherein the sensing layer is hermetically sealed to the coil layer, and wherein the coil layer is hermetically sealed to the integrated circuit layer.

9. The intraocular physiological sensor implant of claim 1, further comprising an anchor, wherein the anchor is either coupled to a housing or integrally formed with the housing.

10. The intraocular physiological sensor implant of claim 9, wherein the anchor is configured to pierce a trabecular meshwork of an eye, such that the intraocular physiological sensor is retained in an anterior chamber of the eye, adjacent to the trabecular meshwork.

11. The intraocular physiological sensor implant of claim 10, wherein the anchor further includes a plurality of physical standoffs.

12. The intraocular physiological sensor implant of claim 1, wherein the physiological sensor comprises a glucose sensor.

13. An intraocular physiological sensor implant comprising:
a physiological sensor, configured to sense a pressure; and
a housing;
wherein the physiological sensor includes a sensing layer, a coil layer, and an integrated circuit layer, and
wherein the physiological sensor is configured to communicate pressure readings to an external reader, responsive to the external reader being held proximate to an eye of a patient.

14. The intraocular physiological sensor implant of claim 13, wherein the physiological sensor comprises a capacitive pressure sensor.

15. The intraocular physiological sensor implant of claim 14, wherein the capacitive pressure sensor comprises a flexible diaphragm electrode spaced apart from a counter electrode.

16. The intraocular physiological sensor implant of claim 13, wherein the coil layer includes a looped coil, configured to communicate with the external reader.

17. The intraocular physiological sensor implant of claim 13, wherein the sensing layer is hermetically sealed to the coil layer, and wherein the coil layer is hermetically sealed to the integrated circuit layer.

18. The intraocular physiological sensor implant of claim 13, further comprising an anchor, wherein the anchor is either coupled to the housing or integrally formed with the housing.

19. The intraocular physiological sensor implant of claim 18, wherein the anchor is configured to pierce a trabecular meshwork of an eye, such that the intraocular physiological sensor is retained in an anterior chamber of the eye, adjacent to the trabecular meshwork.

20. The intraocular physiological sensor implant of claim 19, wherein the anchor further includes a plurality of physical standoffs.
